(19) United States
(12) Patent Application Publication
(10) Pub. No.: US 2019/0099064 A1
(43) Pub. Date: Apr. 4, 2019

(54) FLEXIBLE TUBE INSERTION APPARATUS

(71) Applicant: OLYMPUS CORPORATION, Tokyo (JP)

(72) Inventor: Shuji NAKAMURA, Akishima-shi (JP)

(73) Assignee: OLYMPUS CORPORATION, Tokyo (JP)

(21) Appl. No.: 16/208,672

(22) Filed: Dec. 4, 2018

Related U.S. Application Data
(63) Continuation of application No. PCT/JP2016/067256, filed on Jun. 9, 2016.

(51) Int. Cl. A61B 1/00; A61B 1/005; G02B 23/24

(52) U.S. Cl. CPC A61B 1/00078; A61B 1/00006; A61B 1/008; G02B 23/2476; A61B 1/00147; A61B 1/0051

(57) ABSTRACT

An insertion apparatus includes a flexible tube to be inserted into an insertion target. The flexible tube is divided into segments arranged in series along a central axis. The insertion apparatus also includes a variable stiffness portion that varies a bending stiffness of the flexible tube in units of segments, a detector that detects state information of the flexible tube including velocity information, a calculator that calculates the velocity information of the flexible tube at two portions of the flexible tube based on the state information, and a controller that controls the bending stiffness of the flexible tube in units of segments through the variable stiffness portion based on a velocity ratio calculated based on the velocity information of the two portions.

### CROSS-REFERENCE TO RELATED APPLICATIONS

This application is a Continuation Application of PCT Application No. PCT/JP2016/067256, filed Jun. 9, 2016, the entire contents of which are incorporated herein by reference.

### BACKGROUND OF THE INVENTION

### 1. Field of the Invention

The present invention relates to a flexible tube insertion apparatus that inserts a flexible tube into a tube portion of an insertion target.

### 2. Description of the Related Art

For example, a flexible tube of an insertion section disclosed in Jpn. Pat. Appln. KOKOKU Publication No. S61-37931 is divided into segments arranged in series along a central axis of the flexible tube. The bending stiffness of each segment is capable of being varied. This improves the insertability of the flexible tube into a deeper portion.

For example, an endoscope system disclosed in Jpn. Pat. Appln. KOKAI Publication No. H6-70879 includes an endoscope that controls the flexibility of a flexible tube for each segment, and a database that stores shape information and a degree of the flexibility of the endoscope as a flexibility control pattern. The endoscope system changes the flexibility using the database. This improves the insertability of the flexible tube into a deeper portion.

For example, a flexible tube of an endoscope apparatus disclosed in Jpn. Pat. Appln. KOKAI Publication No. 2016-7434 is divided into segments arranged in series along a central axis of the flexible tube. The endoscope apparatus varies the bending stiffness of the flexible tube to a bending stiffness suitable for insertion in units of segments according to the shape of the flexible tube calculated by the shape calculator. Thereby, the insertability of the flexible tube into a deeper portion of a tube portion (for example, an intestine tract of a large intestine) of an insertion target (for example, a patient) improves when an insertion section is push-operated.

### BRIEF SUMMARY OF THE INVENTION

A flexible tube insertion apparatus includes: a flexible tube that is divided into segments arranged in series along a central axis and is to be inserted into an insertion target; a variable stiffness portion that varies a bending stiffness of the flexible tube in units of segments; a state detector that detects state information related to a state of the flexible tube including at least velocity information of the flexible tube; a state calculator that calculates the velocity information of the flexible tube at two portions of the flexible tube based on the state information; and a stiffness controller that controls the bending stiffness of the flexible tube in units of segments through the variable stiffness portion based on a velocity ratio calculated based on the velocity information of the two portions.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram of a flexible tube insertion apparatus according to an embodiment of the present invention.

FIG. 2 is a diagram for explaining relationships among segments, a state detector, a state calculator, a velocity ratio calculator, an insertability determinator, a stiffness controller, a variable stiffness portion, and an input device.

FIG. 3 is a diagram for explaining an arrangement of a front segment, a rear segment, and a control segment, velocities of the front segment and the rear segment, a velocity ratio thereof, and first and second insertion paths.

FIG. 4 is a diagram for explaining a velocity ratio between the front segment and the rear segment, and the control segment arranged between the front segment and the rear segment.

FIG. 5A is a diagram for explaining a relationship between the velocity ratio and a threshold value that is 1.

FIG. 5B is a diagram for explaining a relationship between the velocity ratio and a threshold value that is 0.8.

FIG. 6 is a diagram for explaining an example of control of the stiffness controller for a control segment.

FIG. 7 is a diagram for explaining a first example of control of the stiffness controller.

FIG. 8 is a diagram for explaining a second example of control of the stiffness controller.

FIG. 9A is a diagram for explaining a third example of control of the stiffness controller.

FIG. 9B is a diagram for explaining a fourth example of control of the stiffness controller.

FIG. 10 is a diagram for explaining a fifth example of control of the stiffness controller.

FIG. 11 is a diagram for explaining a sixth example of control of the stiffness controller.

FIG. 12 is a diagram for explaining a seventh example of control of the stiffness controller.

### DETAILED DESCRIPTION OF THE INVENTION

Hereinafter, an embodiment of the present invention will be described in detail with reference to the drawings. In some of the drawings, illustration of a part of a member is omitted for clarification of the drawings.

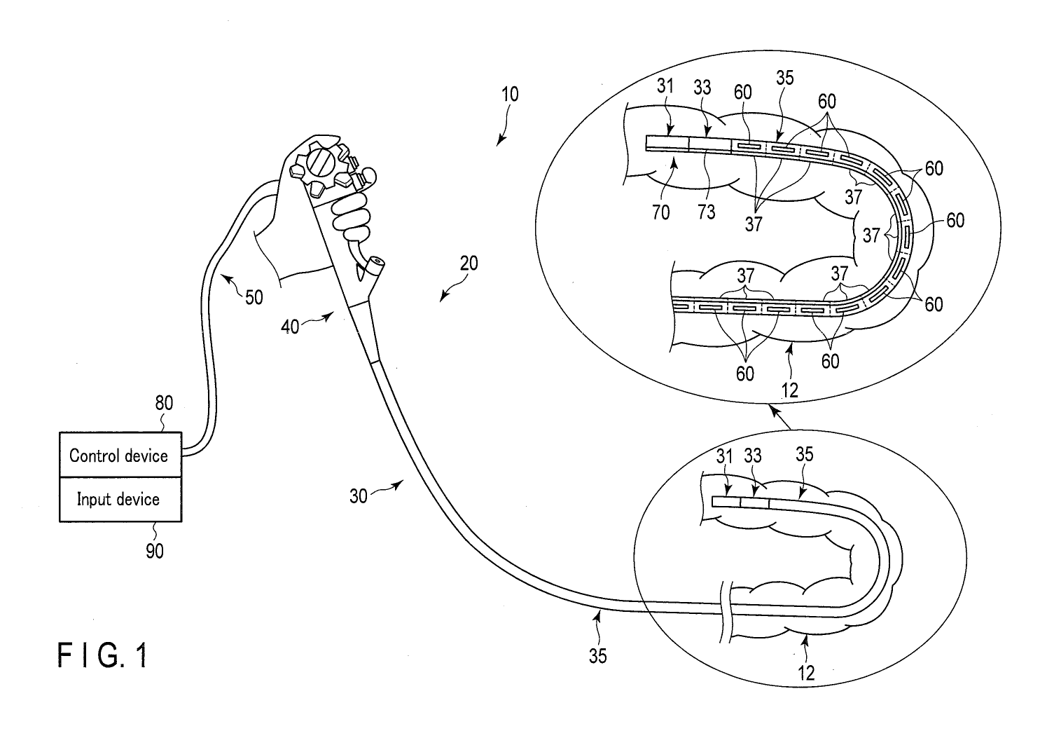

As shown FIG. 1, the flexible tube insertion apparatus (hereinafter, referred to as an insertion apparatus 10) includes an endoscope 20, a control device 80 that controls the endoscope 20, and an input device 90 connected to the control device 80. For example, the control device 80 functions as a stiffness control device that controls bending stiffness of a flexible tube 35 of the insertion section 30 arranged in the endoscope 20. Although not shown, the insertion apparatus 10 may include a display device that displays an image captured by the endoscope 20, and a light source device that emits light for observing and imaging by the endoscope 20.

The endoscope 20 will be described as a medical flexible endoscope, for example, but it is not necessary to be limited thereto. The endoscope 20 is only required to include, for example, a flexible insertion section 30 to be inserted into a tube portion 12 (for example, an intestine tract of a large intestine) of an insertion target (for example, a patient), such as an industrial flexible endoscope, a catheter, and a treatment tool. The insertion section 30 is only required to include a portion (for example, the flexible tube 35, which will be described later) having flexibility so as to be bent by an external force. The endoscope 20 may be a direct view type endoscope or a side view type endoscope. The insertion target is not limited to a person, and may be an animal, or other structures, for example. The tube portion 12 may be an industrial pipe, for example.

The endoscope 20 includes an insertion section 30, a grip portion 40 that is connected to a proximal end of the insertion section 30 and is to be gripped by an operator of the insertion apparatus 10, and a universal cord 50 extending from a side surface of the grip portion 40. The universal cord 50 has a connector (not shown) detachably attached to the control device 80.

The insertion section 30 is tubular, elongated, and flexible. The insertion section 30 is moved back and forth inside the tube portion 12 with respect to the tube portion 12. The insertion section 30 is capable of being bent according to the shape of the tube portion 12. The insertion section 30 includes a distal hard section 31, a bendable section 33, and the flexible tube 35 in order from the distal end of the insertion section 30 toward the proximal end of the insertion section 30. The distal hard section 31 and the bendable section 33 are shorter than the flexible tube 35. Therefore, in the present embodiment, the distal hard section 31, the bendable section 33, and the distal end of the flexible tube 35 are regarded as the distal end of the insertion section 30. The flexible tube 35 has flexibility and is bent by an external force.

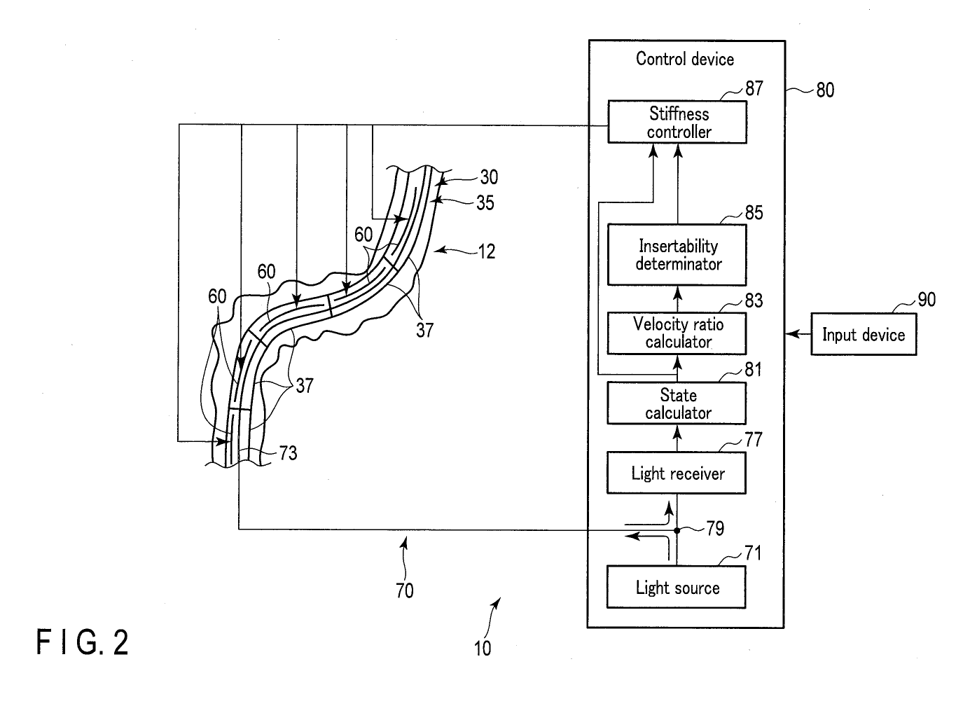

As shown in FIGS. 1 and 2, the flexible tube 35 of the insertion section 30 is divided into segments 37 arranged in series along a central axis of the insertion section 30. For example, it is assumed that the segments 37 are present over an entire length of the flexible tube 35. The segments 37 may be present, for example, in a part of the flexible tube 35. The bending stiffness of each segment 37 is capable of being independently varied by control of the control device 80. Therefore, the bending stiffness of the flexible tube 35 is capable of being partially varied by the bending stiffness of each segment 37 independently controlled by the control device 80. It should be noted that the segment 37 may function as an imaginary virtual region or may function as a real structure. The lengths of the segments 37 may be the same as or different from one another. For example, the length of a portion to be inserted into the insertion target of the insertion section 30 is determined according to the insertion target. Therefore, it may be configured that the portion to be inserted is divided into the segments 37, and a portion disposed outside the insertion target and not inserted into the insertion target is regarded as one segment 37.

The insertion apparatus 10 has one or more variable stiffness portions 60 that have stiffness that is varied by the control of the control device 80 and vary the bending stiffness of the flexible tube 35 by the stiffness. In this embodiment, the variable stiffness portion 60 varies the bending stiffness of the flexible tube 35 in the insertion section 30 in units of segments. Thus, it is assumed that variable stiffness portions 60 are each incorporated into, for example, each segment 37, and are incorporated over the entire length of the flexible tube 35. Therefore, the segments 37 including the variable stiffness portions 60 can function as a control target (hereinafter, referred to as a control segment 37i) that is controlled by a stiffness controller 87, which will be described later, arranged in the control device 80. It should be noted that the variable stiffness portions 60 are only required to be arranged in sites of the flexible tube 35 that are inserted into the tube portion 12 and the bending stiffness of which need to be varied. That is, the variable stiffness portions 60 may be incorporated into only a part of the segments 37.

Portions provided with the variable stiffness portions 60 may function as at least the segments 37. A single variable stiffness portion 60 may be incorporated over some segments 37. The variable stiffness portions 60 may be arranged in a single row along the central axis of the insertion section 30, or may be arranged in multiple rows. In the case where the variable stiffness portions 60 are arranged in multiple rows, variable stiffness portions 60 may be provided at the same position so that the variable stiffness portions 60 are adjacent to one another in a circumferential direction of the flexible tube 35, or may be provided so as to be displaced along the central axis of the insertion section 30.

Although not shown, the variable stiffness portion 60 is constituted by, for example, an actuator including a coil pipe formed of a metal wire and an electroactive polymer artificial muscle (hereinafter, referred to as EPAM) sealed inside the coil pipe. The central axis of the coil pipe is provided coincident with or in parallel to the central axis of the insertion section 30. The coil pipe includes electrodes provided at both ends of the coil pipe.

Each of the electrodes of the variable stiffness portion 60 is connected to the control device 80 through a signal cable incorporated into the endoscope 20, and is supplied with electric power from the control device 80. When a voltage is applied across the EPAM through the electrodes, the EPAM tends to expand or contract along the central axis of the coil pipe. However, the expansion and contraction of the EPAM is restricted by the coil pipe. As a result, the stiffness of the variable stiffness portion 60 varies. The stiffness of the variable stiffness portion 60 increases as a value of the voltage applied increases. When the stiffness of the variable stiffness portion 60 varies, the bending stiffness of the segment 37 varies accordingly. In addition, the electric power is independently supplied to each electrode. Therefore, the stiffness of each of the variable stiffness portions 60 independently varies, and the bending stiffness of each of the segments 37 also varies independently. In this manner, the variable stiffness portion 60 varies the bending stiffness of the segment 37 by stiffness variation of the variable stiffness portion 60, so as to partially vary the bending stiffness of the flexible tube 35 by bending stiffness variation of the segment 37.

A shape-memory alloy may be used instead of the EPAM for the variable stiffness portion 60.

The insertion apparatus 10 includes a state detector 70 that detects state information of the flexible tube 35 including at least velocity information of the flexible tube 35. The velocity information includes a magnitude of the velocity and a direction of the velocity of the flexible tube 35 along the central axis of the flexible tube 35. Further, the state information of the flexible tube 35 includes a bending state of the flexible tube 35. The bending state of the flexible tube 35 includes, for example, a bending quantity (a magnitude of bending) of the flexible tube 35. The bending state of the flexible tube 35 may include a direction of bending of the flexible tube 35.

As an example, the state detector 70 includes a fiber sensor that utilizes a loss of light transmission quantity due to bending of an optical fiber 73. The fiber sensor includes a light source 71 that emits light, a single optical fiber 73 that guides the light, a reflector (not shown) that reflects the light so that the light guided by the optical fiber 73 is reversed in the optical fiber 73, a light receiver 77 that receives the reflected light, and a light branching unit 79. The light source 71 includes, for example, an LED, etc. The light source 71 is separate from a light source of the light source device that emits light for observing and imaging. The optical fiber 73 is incorporated in the endoscope 20, and has flexibility. The optical fiber 73 includes detection targets (not shown) to be mounted on the insertion section 30. The detection targets are arranged in mutually-different positions along a longitudinal axis of the optical fiber 73. The detection targets are only required to be arranged in sites in which the bending stiffness of the flexible tube 35 is to be varied. Accordingly, in the present embodiment, it is assumed that the detection target is arranged in each segment 37 inside the flexible tube 35. The optical fiber 73 and the variable stiffness portions 60 are arranged side by side in the flexible tube 35. The reflector is arranged in a distal end of the optical fiber 73 positioned in the distal end of the insertion section 30. The light receiver 77 may include, for example, an element for spectroscopy such as a spectroscope or a color filter, and a light receiving element such as a photodiode. The light source 71, the light receiver 77, and the proximal end of the optical fiber 73 are optically connected to the light branching unit 79. The light branching unit 79 includes, for example, an optical coupler or a half mirror. The light branching unit 79 guides light emitted from the light source 71 to the optical fiber 73, and guides return light reflected by the reflector and guided by the optical fiber 73 to the light receiver 77. That is, the light travels through the light source 71, the light branching unit 79, the optical fiber 73, the reflector, the optical fiber 73, the light branching unit 79, and the light receiver 77 in this order. The light source 71, the light receiver 77, and the light branching unit 79 are mounted on the control device 80, for example.

When the insertion section 30 is bent, in accordance with this bend, the optical fiber 73 is bent. Along with this, part of the light propagating through the optical fiber 73 is radiated (leaked) to the outside through, for example, the detection targets each having sensitivity to a different wavelength. The detection targets are to change optical characteristics of the optical fiber 73, for example, a light transmission quantity of light at a predetermined wavelength. Therefore, when the optical fiber 73 is bent, the light transmission quantity of the optical fiber 73 is changed in accordance with the bending quantity of the optical fiber 73. An optical signal including information on this change in the light transmission quantity is received by the light receiver 77. The light receiver 77 outputs the optical signal as state information of the flexible tube 35 to the state calculator 81 arranged in the control device 80.

Meanwhile, a single detection target may be arranged in a single optical fiber 73, and in this case, optical fibers are arranged. Then, it is assumed that detection targets are arranged in the same position or in the vicinity along the longitudinal axis of the optical fiber 73, and in mutually-different positions in a direction around the longitudinal axis. In this case, the bending quantity and the direction of bending can be detected by a combination of detection results of the detection targets.

The state detector 70 is not limited to including a fiber sensor. The state detector 70 may include, for example, any one of a distortion sensor, an acceleration sensor, a gyro sensor, and an element such as a coil. The distortion sensor, for example, detects a bending distortion due to an external force (pressure) that the flexible tube 35 receives from the outside (for example, the tube portion 12). The acceleration sensor detects acceleration of the flexible tube 35. The gyro sensor detects an angular velocity of the flexible tube 35. The element generates a magnetic field corresponding to a state of the flexible tube 35, such as the shape of the flexible tube 35.

The state detector 70 always detects (operates) after a detection start instruction is input from the input device 90 to the state detector 70. Note that a timing for the detection maybe such that the detection is executed for every elapse of a predetermined time, and is not limited in particular.

As shown in FIG. 2, the insertion apparatus 10 includes the state calculator 81, the velocity ratio calculator 83, the insertability determinator 85, and the stiffness controller 87. The state calculator 81, the velocity ratio calculator 83, the insertability determinator 85, and the stiffness controller 87 can be arranged in the control device 80. The state calculator 81, the velocity ratio calculator 83, and the insertability determinator 85 always operate after an operation start instruction is input from the input device 90. The stiffness controller 87 always operates after a control start mode, which will be described later, is selected by the input device 90.

The state calculator 81, the velocity ratio calculator 83, the insertability determinator 85, and the stiffness controller 87 are each configured by, for example, a hardware circuit including an ASIC, etc. At least one of the state calculator 81, the velocity ratio calculator 83, the insertability determinator 85, and the stiffness controller 87 maybe configured by a processor. In the case where at least one of these is configured by a processor, a program code for causing the processor to function as at least one of these by being executed by the processor has been stored in an internal memory or an external memory (not shown) accessible by the processor.

Based on the state information detected by the state detector 70, the state calculator 81 calculates the shape information related to the shape of the flexible tube 35 along the central axis of the flexible tube 35. For example, at a predetermined time, the state calculator 81 calculates the shape information of the flexible tube 35 from a relationship between characteristics of light entering the optical fiber 73 and light exiting from the optical fiber 73. In detail, the state calculator 81 calculates the shape information, specifically a bent shape of an actually bent portion of the flexible tube 35, based on the state information output from the fiber sensor. The bent shape of the flexible tube 35 includes, for example, a curvature radius of the flexible tube 35. In addition, the state calculator 81 can calculate a direction of the center of bending of the flexible tube 35 based on the state information (shape information).

The state calculator 81 regards the bent shape of the flexible tube 35 calculated every predetermined time as an insertion path of the flexible tube 35 in an insertion process. For this purpose, the state calculator 81 calculates the shape information of each segment 37 based on the state information. Then, the state calculator 81 calculates the shape information of the flexible tube 35 by joining the shape information of each segment 37, to regard the calculated shape information as the insertion path of the flexible tube 35. The state calculator 81 calculates the insertion path for each time.

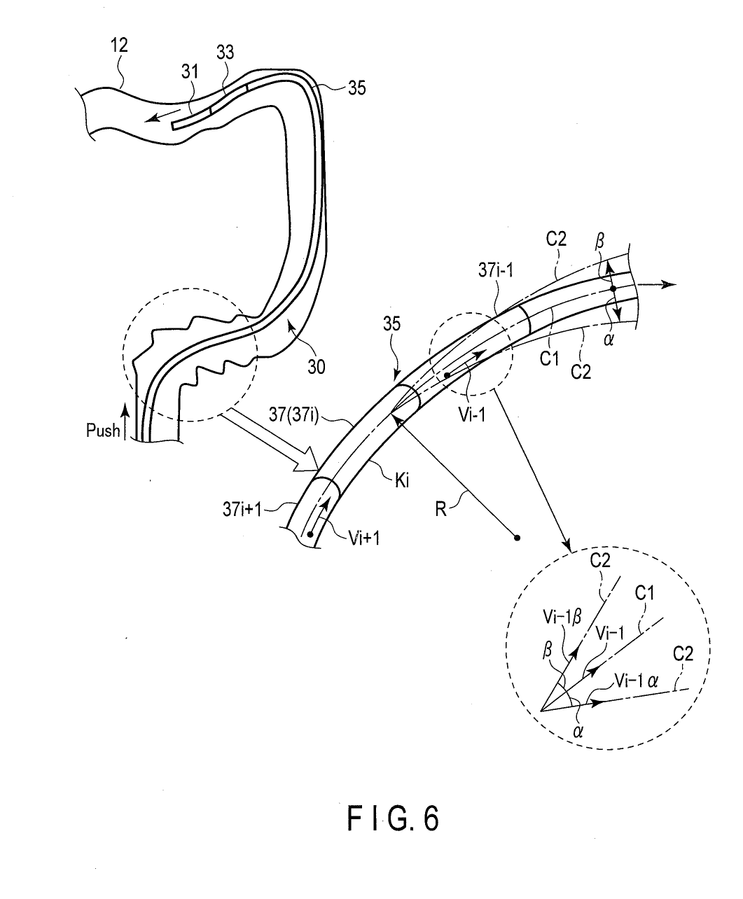

Here, the shape information calculated by the state calculator 81 at the first and second times are referred to as first and second shape information, respectively. The second time is later than the first time. The insertion paths at the first and second times are referred to as first and second insertion paths C1 and C2, respectively (see FIG. 6). Based on the first shape information and the second shape information, the state calculator 81 also calculates information related to a change in shape of the segment 37 between the first time and the second time as the shape information. Since the shape information indicates the insertion path of the flexible tube 35, the shape change indicates a displacement quantity (bending angle) of the second insertion path C2 with respect to the first insertion path C1. The shape change indicates whether a segment 37 bends toward the center direction of previous bending of the segment 37 (see an angle a shown in FIG. 6) or bends in a direction opposite to the center direction of previous bending of the segment 37 (see an angle β shown in FIG. 6). Bending in the center direction means, for example, that a curvature radius at a time T2 is smaller than a curvature radius at a time T1. Bending in the direction opposite to the center direction means, for example, that the curvature radius at time T2 is larger than the curvature radius at time T1.

Based on the state information of the flexible tube 35 detected by the state detector 70, the state calculator 81 calculates the velocity information of the flexible tube 35 at two portions of the flexible tube 35. The state calculator 81, for example, differentiates position information in the state information of the flexible tube 35 obtained from the fiber sensor or from the element to calculate the velocity information. Alternatively, the state calculator 81 may, for example, integrate the acceleration in the state information of the flexible tube 35 obtained from the acceleration sensor to calculate the velocity information.

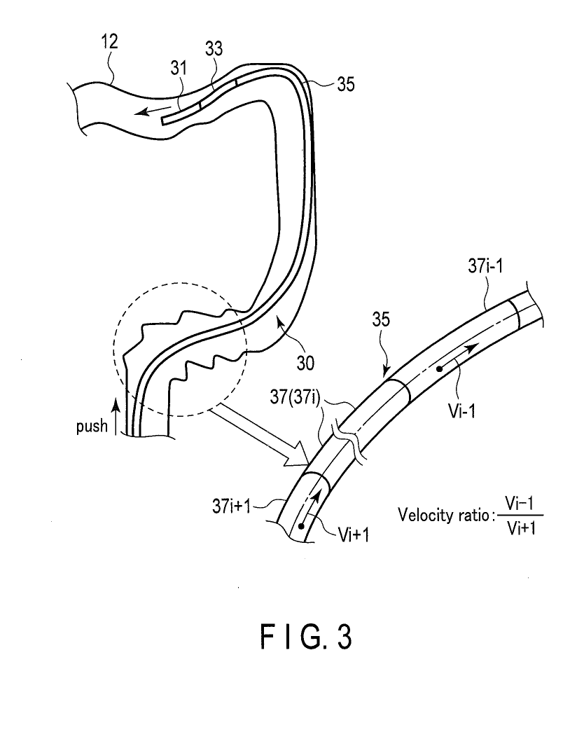

The two portions indicate, for example, two segments 37 arranged to be spaced apart from each other, not two portions in a segment 37. Here, these two segments 37 are referred to as segments 37i−1 and 37i+1 as shown in FIG. 3. It is assumed that the segment 37i−1 is arranged in front of the segment 37i+1 in the insertion direction of the flexible tube 35. Therefore, the segment 37i−1 is a front portion (front segment), and the segment 37i+1 is a rear portion (rear segment). The segment 37i−1 and the segment 37i+1 are set by the input device 90, and then information on them are input to the state calculator 81 from the input device 90.

The velocity information indicates magnitudes and directions of respective velocities Vi−1 and Vi+1 of the segments 37i−1 and 37i+1 along the central axis of the flexible tube 35. The velocity information may be stored in a storage (not shown).

The two portions are not limited to the segments 37i−1 and 37i+1 as long as the velocity information can be detected. For example, the two portions may indicate a front portion arranged in front in the insertion direction of the flexible tube 35, and a rear portion arranged rearward with respect to the front portion in the insertion direction of the flexible tube 35 and arranged to be spaced apart from the front portion. In this case, the velocity information indicates, for example, the magnitude and the direction of the velocity Vi−1 of the front portion and the magnitude and the direction of the velocity Vi+1 of the rear portion.

In the present embodiment, segments 37 arranged between the segment 37i−1 and the segment 37i+1 functions as a control segment 37i. The control segment 37i is all the segments 37 arranged between the segment 37i−1 and the segment 37i+1. The control segment 37i may be any one of the segments 37 arranged between the segment 37i−1 and the segment 37i+1. As such, the number of the control segments 37i is equal to or more than 1. Therefore, the segment 37i−1 is arranged in front of the control segment 37i, and the segment 37i+1 is arranged behind the control segment 37i. In the present embodiment, at least three segments 37 are required.

The state calculator 81 outputs the calculated insertion path for each time and the calculated velocity information for each time to the velocity ratio calculator 83.

The velocity ratio calculator 83 calculates a velocity ratio based on the velocity information at the two portions calculated by the state calculator 81. The velocity ratio is a value obtained by dividing a velocity of the distal end side of the flexible tube 35 by a velocity of the proximal end (hand) side of the flexible tube 35. In the present embodiment, the velocity ratio indicates a value obtained by dividing the velocity Vi−1 of the segment 37i−1, which is the front segment arranged forward in the insertion direction of the flexible tube 35, by the velocity Vi+1 of the segment 37i+1, which is the rear segment arranged rearward in the insertion direction of the flexible tube 35. That is, the velocity ratio calculator 83 calculates the velocity ratio (Vi−1/Vi+1) based on the velocities Vi−1 and Vi+1 of the segments 37i−1 and 37i+1. The velocity ratio calculator 83 outputs the calculated velocity ratio to the insertability determinator 85.

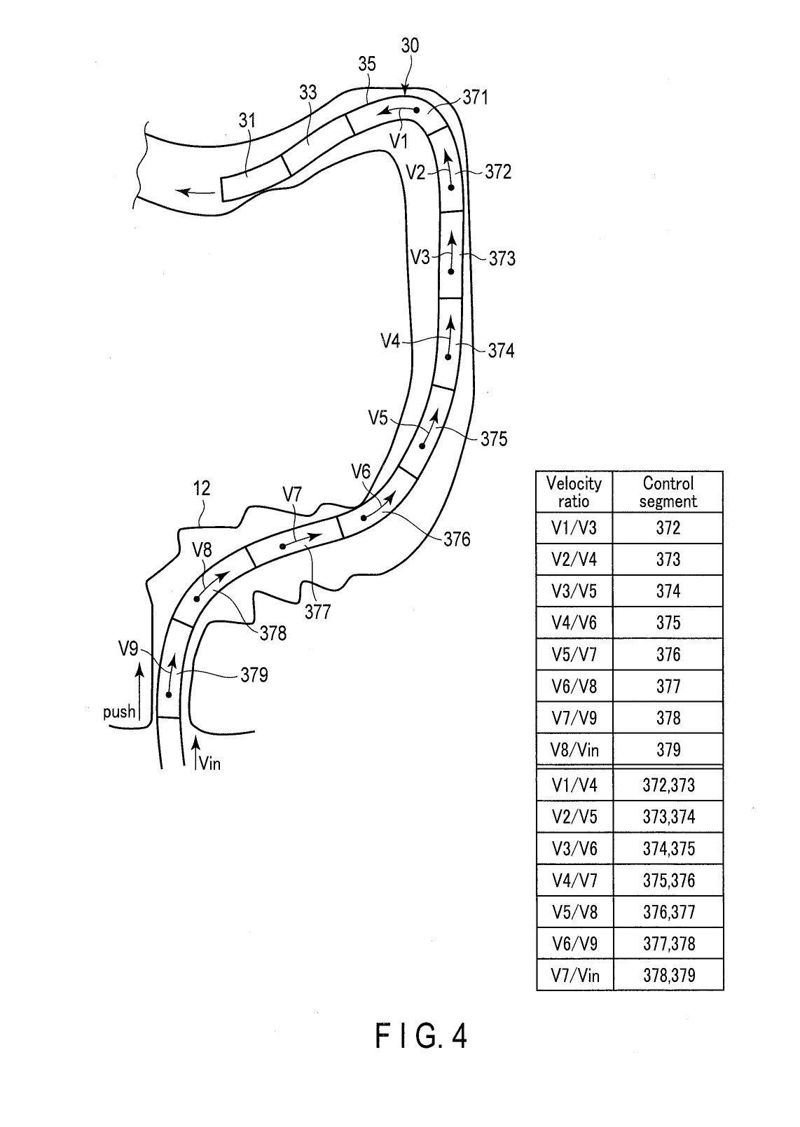

Herein, as shown in FIG. 4, it is assumed that the segments 371, 372, 373, 374, 375, 376, 377, 378, and 379 are arranged in this order from the front side to the rear side in the insertion direction of the flexible tube 35, and the respective velocities along the central axis of the flexible tube 35 are referred to as velocities V1, V2, V3, V4, V5, V6, V7, V8, and V9. The velocity on the hand side of the flexible tube 35 along the central axis of the flexible tube 35 is referred to as Vin. The velocity Vin is a velocity of a gripped portion of the flexible tube 35 gripped by an operator. For example, when the segments 371 and 373 are set by the input device 90 as the segments 37i−1 and 37i+1 to be used by the state calculator 81 and the velocity ratio calculator 83, the velocity ratio is V1/V3, and the control segment 37i is the segment 372. For example, when the segments 372 and 374 are set, the velocity ratio is V2/V4, and the control segment 37i is the segment 373. When the segment 378 and the hand side are set, the velocity ratio is V8/Vin, and the control segment 37i is the segment 379. For example, when the segments 371 and 374 are set, the velocity ratio is V1/V4, and the control segment 37i is the segments 372 and 373. When the segments 372 and 375 are set, the velocity ratio is V2/V5, and the control segment 37i is the segments 373 and 374. When the segment 377 and the hand side are set, the velocity ratio is V7/Vin, and the control segment 37i is the segments 378 and 379.

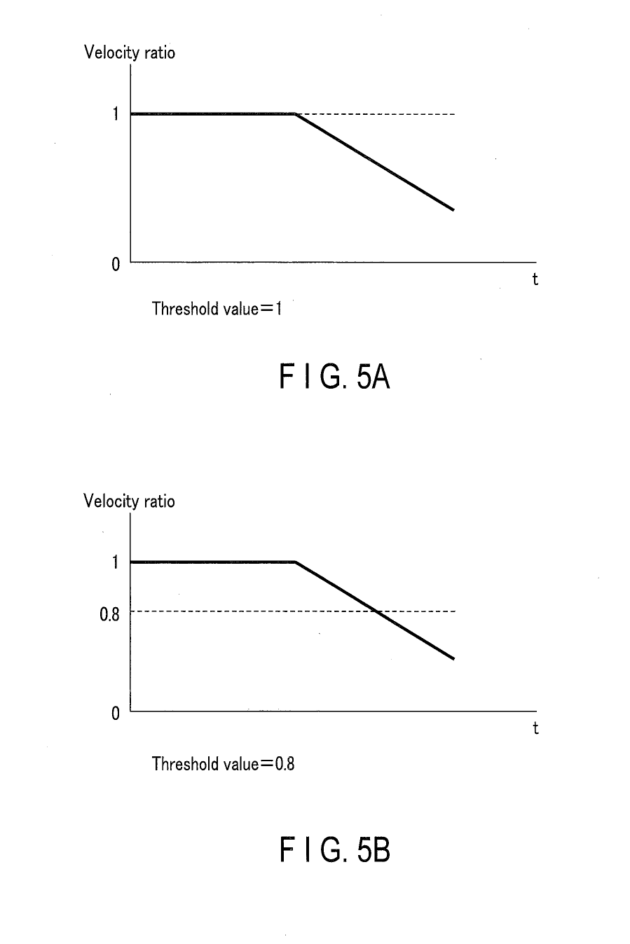

The insertability determinator 85 determines whether the insertability of the flexible tube 35 from a current position to a deeper portion is decreased based on the velocity ratio calculated by the velocity ratio calculator 83 and a threshold value. As shown in FIGS. 5A and 5B, for example, the insertability determinator 85 compares the velocity ratio with the threshold value to determine that the insertability is decreased when the velocity ratio is less than the threshold value (for example, 1, 0.8, etc.). If the velocity ratio is equal to or more than the threshold value, the insertability determinator 85 determines that the insertability is not decreased. The insertability determinator 85 may determine that the insertability is decreased when the velocity ratio is less than the threshold value and a temporal change of the velocity ratio is negative. The threshold value to be used in the insertability determinator 85 is set by the input device 90, and is input from the input device 90 to the insertability determinator 85. The insertability determinator 85 outputs a determination result to the stiffness controller 87.

The velocity ratio calculated by the velocity ratio calculator 83 and the determination result of the insertability determinator 85 may be displayed on a display device. The velocity ratio and the determination result may be announced to the operator by sound, etc.

The stiffness controller 87 controls the bending stiffness of the flexible tube 35 in units of segments through the variable stiffness portion 60 based on the velocity ratio calculated based on the velocity information of the two portions. Specifically, when the insertability determinator 85 determines that the insertability of the flexible tube 35 is decreased, the stiffness controller 87 controls the bending stiffness in units of segments so that the velocity ratio calculated by the velocity ratio calculator 83 becomes a target value of the velocity ratio. At this time, the stiffness controller 87 controls the bending stiffness of the control segment 37i. Furthermore, the stiffness controller 87 controls the bending stiffness of the control segment 37i to be high or low based on the shape change calculated by the state calculator 81. For example, when the segment 37i−1 is bent towards the center direction of the previous bending of the segment 37i−1, the stiffness controller 87 controls the bending stiffness of the control segment 37i to be high. For example, when the segment 37i−1 is bent in a direction opposite to the center direction of the previous bending of the segment 37i−1, the stiffness controller 87 controls the bending stiffness of the control segment 37i to be low.

As described above, the control segment 37i is one or more segments 37 arranged between the segment 37i−1 and the segment 37i+1 used by the state calculator 81 and the velocity ratio calculator 83. Therefore, the stiffness controller 87 controls the bending stiffness of each of one or more of the segments 37 arranged between the segment 37i−1, which is the front segment, and the segment 37i+1, which is the rear segment, so that the velocity ratio becomes a target value of the velocity ratio.

The stiffness controller 87 comprises a control start mode including, for example, an automatic start mode and a manual start mode. The automatic start mode or the manual start mode is selected through the input device 90.

In the automatic start mode, the stiffness controller 87 controls the bending stiffness of the flexible tube 35 through the variable stiffness portion 60 immediately after the insertability determinator 85 determines that the insertability of the flexible tube 35 is decreased. Specifically, when a determination result that the velocity ratio is less than the threshold value is input from the insertability determinator 85 to the stiffness controller 87, the stiffness controller 87 starts control immediately.

In the manual start mode, when the insertability determinator 85 determines that the insertability of the flexible tube 35 is decreased and the input device 90 inputs a control start instruction to the stiffness controller 87, the stiffness controller 87 controls the bending stiffness of the flexible tube 35 through the variable stiffness portion 60. In further detail, when the determination result that the velocity ratio is less than the threshold value is input from the insertability determinator 85 to the stiffness controller 87 and the input device 90 inputs the control start instruction to the stiffness controller 87, the stiffness controller 87 starts control.

The input device 90 is a general input device, for example, a keyboard, a pointing device such as a mouse, a tag reader, a button switch, a slider, or a dial. The input device 90 may be used by the operator to input various commands to operate the insertion apparatus 10.

The input device 90 sets a front segment and a rear segment to be used in a calculation operation of the state calculator 81, a threshold value in the insertability determinator 85, and a target value used for a control operation of the stiffness controller 87. The input device 90 sets the target value to be a value equal to or less than 1. The input device 90 inputs the set front segment and rear segment to the state calculator 81, inputs the set threshold value to the insertability determinator 85, and inputs the set target value to the stiffness controller 87. The setting, input, and selection of the control start mode are performed before the flexible tube 35 is inserted into the insertion target, or when the flexible tube 35 is inserted into the insertion target. The setting, input, and selection of the control start mode may be changed while the flexible tube 35 is inserted into the insertion target. It may be configured that the setting contents input by the input device 90 are stored in a storage (not shown), and at the time of operation, the state calculator 81, the insertability determinator 85, and the stiffness controller 87 access the storage to read the setting contents. In addition, the front segment, rear segment, threshold value, and target value may be preset and stored in the storage. In the case where the control segment 37i is one of the segments 37 arranged between the segment 37i−1 and the segment 37i+1, the input device 90 selects the control segment 37i from the segments 37 arranged between the segment 37i−1 and the segment 37i+1.

Herein, with reference to FIG. 6, an example of control of the stiffness controller 87 for a control segment 37i will be described.

At a certain time t1, it is assumed that the flexible tube 35 is being inserted into the tube portion 12. The bending stiffness of one control segment 37i at the time t1 is referred to as Ki. At the time t1, the state calculator 81 calculates the first insertion path C1 of the flexible tube 35 based on the state information of the flexible tube 35 detected by the state detector 70. The first insertion path C1 at the time t1 is an original insertion path as a reference. At the time t1, the state calculator 81 calculates the velocity information of the segments 37i−1 and 37i+1 along the central axis of the flexible tube 35. Herein, the velocities of the segments 37i−1 and 37i+1 along the central axis of the flexible tube 35 are referred to as velocities Vi−1 and Vi+1. The velocities Vi−1 and Vi+1 herein are velocities along the shape of the flexible tube 35 prior to the change of the flexible tube 35, in other words, velocities along the central axis of the original first insertion path C1. At the time t1, the velocity ratio calculator 83 calculates a velocity ratio (Vi−1/Vi+1) based on the velocity Vi−1 and the velocity Vi+1. The velocity ratio (Vi−1/Vi+1) calculated at the time t1 is assumed to be 1 that is a threshold value.

It is assumed that the flexible tube 35 is inserted into a deeper portion at a time t2 after the time t1, and an insertion environment such as a state of contact of the flexible tube 35 with respect to an inner peripheral wall of the tube portion 12 changes. At the time t2 as well, the state calculator 81 and the velocity ratio calculator 83 are driven, and the second insertion path C2, a velocity, and a velocity ratio at the time t2 are calculated.

A case will be considered where, at the time t2, the segment 37i−1 is bent by an angle a toward a center direction of bending R of the flexible tube 35 (segment 37i−1) with respect to the first insertion path C1 due to the change in insertion environment, and after the bending, the velocity Vi−1 of the segment 37i−1 along the central axis of the flexible tube 35 changes to a velocity Vi−1α. This change in bending indicates that a current bending stiffness of the segment 37i−1 is insufficient due to the change in insertion environment. Herein, the bending angle α indicates a bending angle of the second insertion path C2 at the time t2 with respect to the first insertion path C1 at the time t1. The bending angle α is calculated by the state calculator 81 based on the insertion paths C1 and C2 at the times t1 and t2. The bending angle α is output from the state calculator 81 to the stiffness controller 87. The velocity Vi−1α at the time t2 is calculated by the state calculator 81.

The bending angle α and the change in velocity indicate that the bending stiffness of the segment 37i−1 at the time t2 is lower than the bending stiffness of the segment 37i−1 inserted along the first insertion path C1 due to the change in insertion environment. When the segment 37i−1 is bent by the bending angle α and the velocity Vi−1 of the segment 37i−1 changes to the velocity Vi−1α, the state calculator 81 calculates the velocity Vi−1 along the central axis of the flexible tube 35 at the time t2 based on the velocity Vi−1α and the bending angle α. The calculated velocity Vi−1 is Vi−1α·cosα. When comparing the velocity Vi−1 along the central axis of the flexible tube 35 at the time t1 and the velocity Vi−1α·cosα along the central axis of the flexible tube 35 at the time t2, Vi−1>Vi−1α·cosα holds true. Herein, it is assumed that the velocity of the segment 37i+1 at the time t2 remains the velocity Vi+1 of the segment 37i+1 at the time t1. At the time t2, the velocity ratio calculator 83 calculates a velocity ratio (Vi−1α·cosα/Vi+1) based on the velocity Vi−1α·cosα and the velocity Vi+1. Since the velocity Vi−1α·cosα is slower than the velocity Vi−1, the velocity ratio (Vi−1α·cosα/Vi+1) becomes smaller than 1, which is the threshold value, at the time t2. Then, the insertability determinator 85 determines that the insertability of the flexible tube 35 is decreased, and then outputs the determination result to the stiffness controller 87. In order to improve the insertability of the flexible tube 35, the segment 37i−1 arranged along the second insertion path C2 at the time t2 needs to be arranged along the first insertion path C1. In other words, the segment 37i−1 deviating from the first insertion path C1 needs to return to the first insertion path C1. Accordingly, when the segment 37i−1 is bent by the bending angle α toward the center direction of the bending R of the flexible tube 35 (segment 37i−1) with respect to the first insertion path C1, the stiffness controller 87 controls the bending stiffness Ki of the control segment 37i to be high. As a result, the segment 37i−1 returns to the first insertion path C1, and the insertability of the flexible tube 35 is improved.

A case will be considered where, conversely, at the time t2, the segment 37i−1 is bent toward a direction opposite to the center direction of the bending R of the flexible tube 35 (segment 37i−1) with respect to the first insertion path C1 by an angle β due to the change in insertion environment, and after the bending, the velocity Vi−1 of the segment 37i−1 along the central axis of the flexible tube 35 changes to a velocity Vi−1β. This change in bending indicates that the current bending stiffness of the segment 37i−1 is excessive due to the change in insertion environment. Herein, the bending angle β indicates a bending angle of the second insertion path C2 at the time t2 with respect to the first insertion path C1 at the time t1. The bending angle β is calculated by the state calculator 81 based on the insertion paths C1 and C2 at the times t1 and t2. The bending angle β is output from the state calculator 81 to the stiffness controller 87. The velocity Vi−1β at the time t2 is calculated by the state calculator 81.

The bending angle β and the change in velocity indicate that the bending stiffness of the segment 37i−1 at the time t2 is higher than the bending stiffness of the segment 37i−1 inserted along the first insertion path C1 due to the change in insertion environment. When the segment 37i−1 is bent at the bending angle β and the velocity Vi−1 of the segment 37i−1 is changed to the velocity Vi−1β, the state calculator 81 calculates the velocity Vi−1 along the central axis of the flexible tube 35 at the time t2 based on the velocity Vi−1β and the bending angle β. The calculated velocity Vi−1 is Vi−1β·cosβ. When comparing the velocity Vi−1 along the central axis of the flexible tube 35 at the time t1 and the velocity Vi−1β·cosβ along the central axis of the flexible tube 35 at the time t2, Vi−1>Vi−1β·cosβ holds true. Herein, it is assumed that the velocity of the segment 37i+1 at the time t2 remains the velocity Vi+1 of the segment 37i+1 at the time t1. At the time t2, the velocity ratio calculator 83 calculates a velocity ratio (Vi−1β·cos β/Vi+1) based on the velocity Vi−1β·cosβ and the velocity Vi+1. Since the velocity Vi−1β·cosβ is slower than the velocity Vi−1, the velocity ratio (Vi−1β·cosβ/Vi+1) becomes smaller than 1, which is the threshold value, at the time t2. Then, the insertability determinator 85 determines that the insertability of the flexible tube 35 is decreased, and then outputs the determination result to the stiffness controller 87. In order to improve the insertability of the flexible tube 35, the segment 37i−1 arranged along the second insertion path C2 at the time t2 needs to be arranged along the first insertion path C1. In other words, the segment 37i−1 deviating from the first insertion path C1 needs to return to the first insertion path C1. Therefore, when the segment 37i−1 is bent in a direction opposite to the center direction of the bending R of the flexible tube 35 (segment 37i−1) with respect to the first insertion path C1 by the bending angle β, the stiffness controller 87 controls the bending stiffness Ki of the control segment 37i to be low. As a result, the segment 37i−1 returns to the first insertion path C1, and the insertability of the flexible tube 35 is improved.

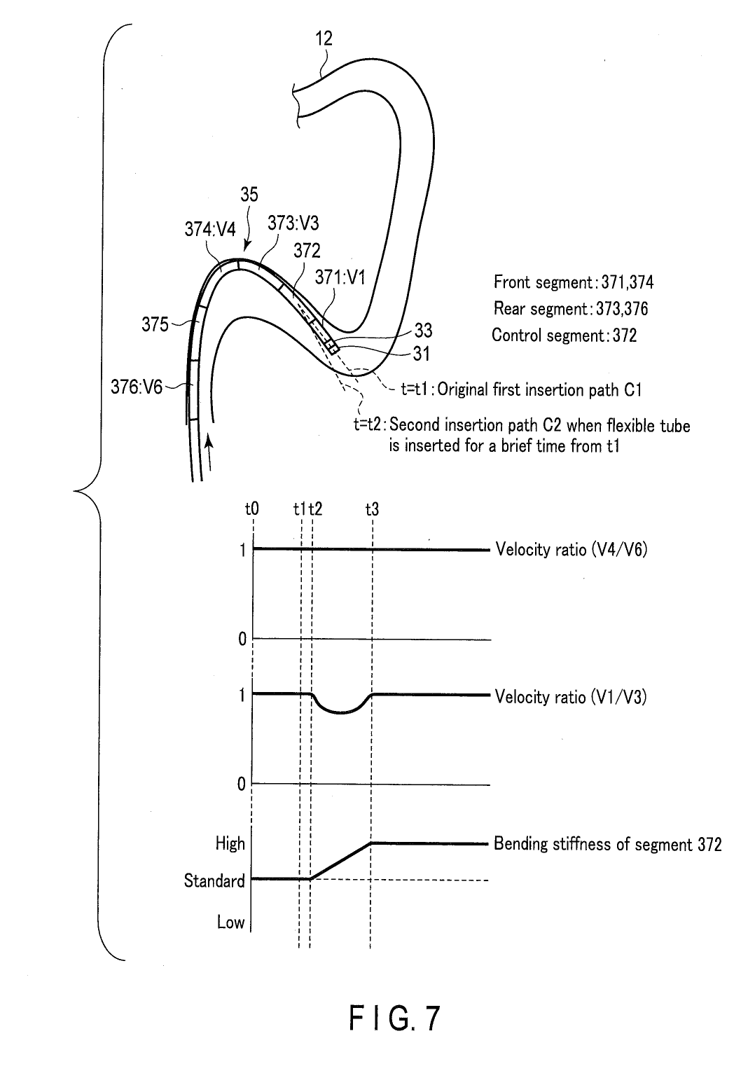

With reference to FIGS. 7 to 11, an example of control of the stiffness controller 87 to be applied to the flexible tube 35 inserted into the tube portion 12 will be described. It is assumed herein that the control segment 37i is a single segment. When the control is actually applied to the flexible tube 35, after the insertion apparatus 10 starts driving, the input device 90 inputs settings of a front segment and a rear segment, a threshold value (for example, 1) of a velocity ratio, a target value (for example, 1) of the velocity ratio, and setting of a control start mode of the stiffness controller 87, to the state calculator 81, to the insertability determinator 85, and to the stiffness controller 87.

It is assumed herein that the segments 371 and 373 are set as the front segment and the rear segment. Accordingly, the control segment 37i is the segment 372. The velocities of the segments 371 and 373 are referred to as V1 and V3, respectively. For reference, the segments 374 and 376 on the hand side and the velocities V4 and V6 of the segments 374 and 376 are also used in the explanation.

The input device 90 outputs an operation start instruction to the insertion apparatus 10. Then, the state detector 70 always detects the state information of the flexible tube 35, and the state calculator 81 always calculates the velocities V1, V3, V4, and V6 in the velocity information of the flexible tube 35 based on the state information of the flexible tube 35. Based on the shape change (a change of the insertion path C2 with respect to the insertion path C1) in the state information of the flexible tube 35, the state calculator 81 calculates whether the segment 371 as the front segment has been bent toward the center direction of the previous bending of the segment 371 or in a direction opposite to the center direction of the previous bending of the segment 371. Then, the state calculator 81 outputs the calculation result to the stiffness controller 87. That is, the state calculator 81 calculates a bending angle to output the calculated bending angle to the stiffness controller 87. In addition, the velocity ratio calculator 83 always calculates the velocity ratios (V1/V3, V4/V6), and the insertability determinator 85 always determines whether the insertability is decreased. The stiffness controller 87 drives in accordance with the control start mode. In the first to fifth examples of control, which will be described below, it is assumed that V4/V6 maintains the target value and the threshold value of the velocity ratio. The insertion path at the time t1 is assumed to be the original first insertion path C1. In addition, the insertion path at the time t2 is assumed to be the second insertion path C2 when the flexible tube 35 is inserted for a very brief time from the time t1.

In the first example of the control shown in FIG. 7, it is assumed that since the bending stiffness of the segment 371 is insufficient at the time t2 with respect to the time t1, the segment 371 is bent toward the center direction of the bending R of the segment 371. That is, at the time t2, the segment 371 deviates from the first insertion path C1, so as to be arranged on the second insertion path C2. In the first example, it is assumed that the input device 90 sets the threshold value of the velocity ratio to 1, sets the target value of the velocity ratio to 1, and sets the control start mode to the automatic start mode. It is also assumed that the tube portion 12 has a single bent portion.

t=t1: The insertability determinator 85 determines that since the velocity ratio (V1/V3) is the same as the threshold value, the insertability is not decreased.

t=t2: The insertability determinator 85 determines that since the velocity ratio (V1/V3) is less than 1, which is the threshold value, the insertability is decreased, to output the determination result to the stiffness controller 87. When the determination result is input, the stiffness controller 87 immediately controls the bending stiffness of the segment 372 to be high so that the velocity ratio (V1/V3) becomes 1, which is the target value. Specifically, the stiffness controller 87 controls the variable stiffness portion 60 arranged in the segment 372 so that the bending stiffness of the segment 372 increases. By this control, the segment 372 becomes hard. As a result, the segment 371 returns from the second insertion path C2 to the first insertion path C1, and thus the insertability of the flexible tube 35 is improved.

t=t3: Since the velocity ratio (V1/V3) has become 1, which is the target value, the stiffness controller 87 controls the variable stiffness portion 60 arranged in the segment 372 so that the high bending stiffness of the segment 372 is maintained.

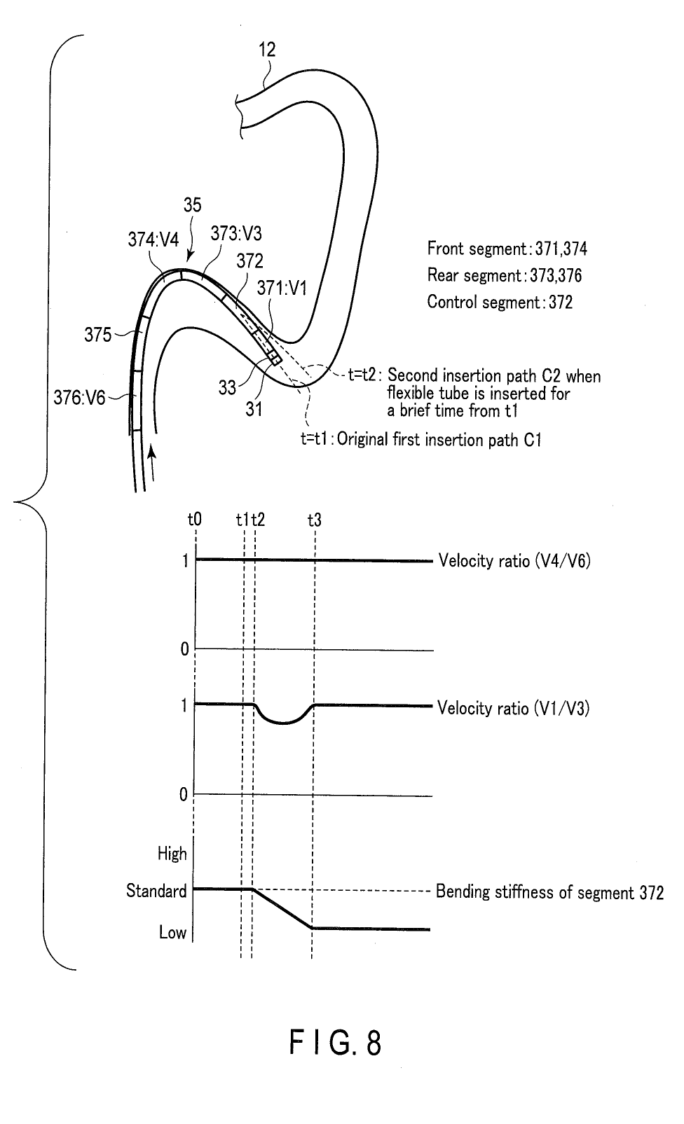

In the second example of the control shown in FIG. 8, it is assumed that since the bending stiffness of the segment 371 is excessive at the time t2 with respect to the time t1, the segment 371 is bent in a direction opposite to the center direction of the bending R of the segment 371. That is, at the time t2, the segment 371 deviates from the first insertion path C1, so as to be arranged on the second insertion path C2. In the second example, a value of the bending stiffness to be controlled is different from that of the first example. In the second example, the input device 90 sets the threshold value of the velocity ratio to 1, sets the target value of the velocity ratio to 1, and sets the control start mode to the automatic start mode. It is also assumed that the tube portion 12 has a single bent portion.

t=t1: The insertability determinator 85 determines that since the velocity ratio (V1/V3) is the same as the threshold value, the insertability is not decreased.

t=t2: The insertability determinator 85 determines that since the velocity ratio (V1/V3) is less than 1, which is the threshold value, the insertability is decreased, to output the determination result to the stiffness controller 87. When the determination result is input, the stiffness controller 87 controls the bending stiffness of the segment 372 to be low so that the velocity ratio (V1/V3) becomes 1, which is the target value. Specifically, the stiffness controller 87 controls the variable stiffness portion 60 disposed in the segment 372 so that the bending stiffness of the segment 372 decreases. By this control, the segment 372 becomes soft. As a result, the segment 371 returns from the second insertion path C2 to the first insertion path C1, and thus the insertability of the flexible tube 35 is improved.

t=t3: Since the velocity ratio (V1/V3) has become 1, which is the target value, the stiffness controller 87 controls the variable stiffness portion 60 arranged in the segment 372 so that the low bending stiffness of the segment 372 is maintained.

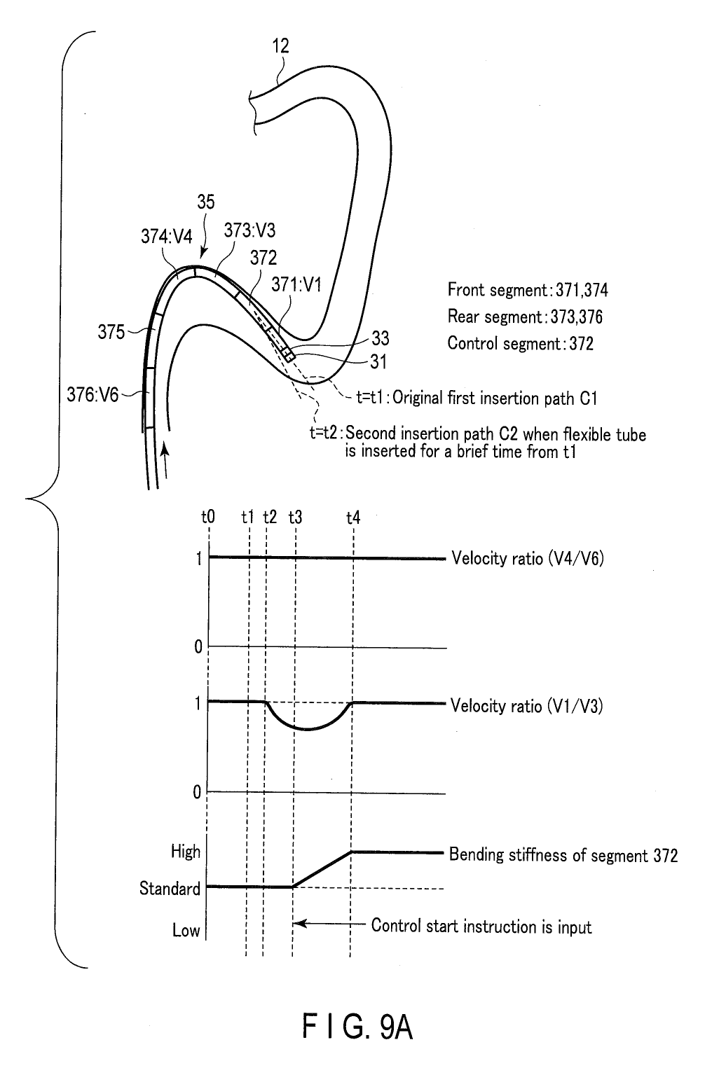

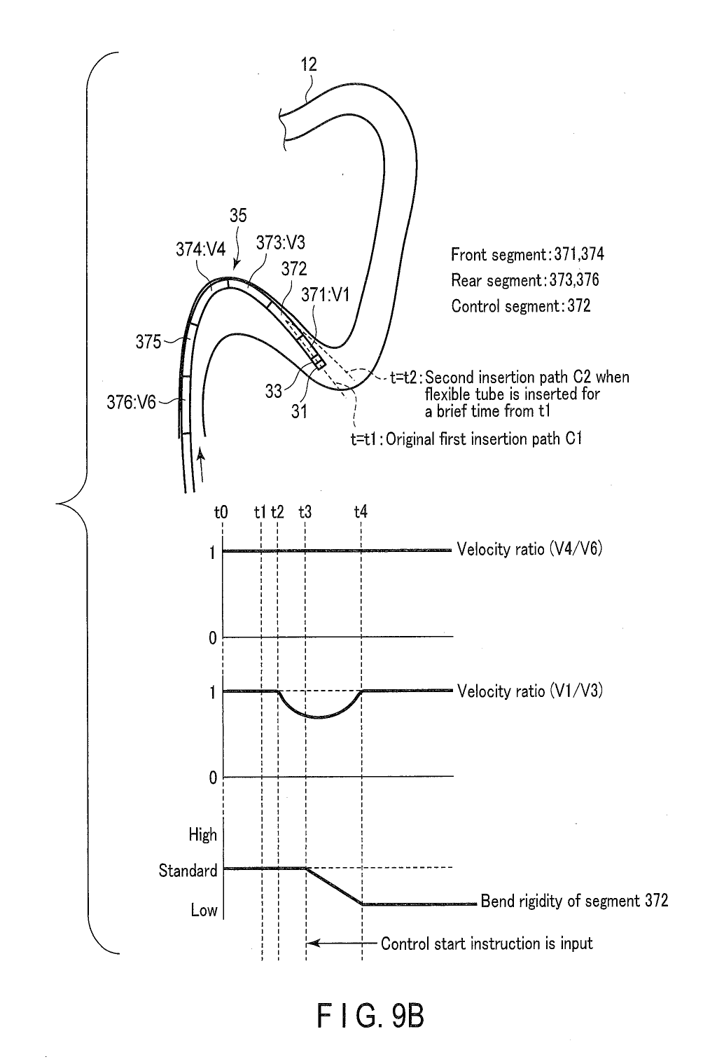

In the third example of the control shown in FIG. 9A, it is assumed that since the bending stiffness of the segment 371 is insufficient at the time t2 with respect to the time t1, the segment 371 is bent toward the center direction of the bending R of the segment 371. In the third example, the control start mode is different from that in the first example. In the third example, the input device 90 sets the threshold value of the velocity ratio to 1, sets the target value of the velocity ratio to 1, and sets the control start mode to the manual start mode. It is also assumed that the tube portion 12 has a single bent portion.

t=t1: The insertability determinator 85 determines that since the velocity ratio (V1/V3) is the same as the threshold value, the insertability is not decreased.

t=t2: The insertability determinator 85 determines that since the velocity ratio (V1/V3) is less than 1, which is the threshold value, the insertability is decreased. For example, the determination result is displayed on the display device. While the operator does not input a control start instruction to the stiffness controller 87 through the input device 90, the stiffness controller 87 does not control the bending stiffness of the segment 372 at the time t2.

t=t3: The operator confirms the determination result displayed on the display device, and the operator inputs the control start instruction to the stiffness controller 87 through the input device 90. Then, the stiffness controller 87 increases the bending stiffness of the segment 372 so that the velocity ratio (V1/V3) becomes 1, which is the target value. Specifically, the stiffness controller 87 controls the variable stiffness portion 60 arranged in the segment 372 so as to increase the bending stiffness of the segment 372. By this control, the segment 372 becomes hard. As a result, the segment 371 returns from the second insertion path C2 to the first insertion path C1, and thus the insertability of the flexible tube 35 is improved. Note that the operator may input the control start instruction to the stiffness controller 87 through the input device 90 according to an operation status at hand, etc.

t=t4: Since the velocity ratio (V1/V3) has become 1, which is the target value, the stiffness controller 87 controls the variable stiffness portion 60 arranged in the segment 372 so that the high bending stiffness of the segment 372 is maintained.

In the fourth example of the control shown in FIG. 9B, it is assumed that since the bending stiffness of the segment 371 is excessive at the time t2 with respect to the time t1, the segment 371 is bent in a direction opposite to the center direction of the bending R of the segment 371. In the fourth example, the control start mode is different from that in the second example. In the fourth example, the input device 90 sets the threshold value of the velocity ratio to 1, sets the target value of the velocity ratio to 1, and sets the control start mode to the manual start mode. It is also assumed that the tube portion 12 has a single bent portion.

t=t1: The insertability determinator 85 determines that since the velocity ratio (V1/V3) is the same as the threshold value, the insertability is not decreased.

t=t2: The insertability determinator 85 determines that since the velocity ratio (V1/V3) is less than 1, which is the threshold value, the insertability is decreased. For example, the determination result is displayed on the display device. While the operator does not input a control start instruction to the stiffness controller 87 through the input device 90, the stiffness controller 87 does not control the bending stiffness of the segment 372 at the time t2.

t=t3: The operator confirms the determination result displayed on the display device, and the operator inputs the control start instruction to the stiffness controller 87 through the input device 90. Then, the stiffness controller 87 decreases the bending stiffness of the segment 372 so that the velocity ratio (V1/V3) becomes 1, which is the target value. Specifically, the stiffness controller 87 controls the variable stiffness portion 60 arranged in the segment 372 so as to decrease the bending stiffness of the segment 372. By this control, the segment 372 becomes soft. As a result, the segment 371 returns from the second insertion path C2 to the first insertion path C1, and thus the insertability of the flexible tube 35 is improved. Note that the operator may input the control start instruction to the stiffness controller 87 through the input device 90 according to an operation status at hand, etc.

t=t4: Since the velocity ratio (V1/V3) has become 1, which is the target value, the stiffness controller 87 controls the variable stiffness portion 60 arranged in the segment 372 so that the low bending stiffness of the segment 372 is maintained.

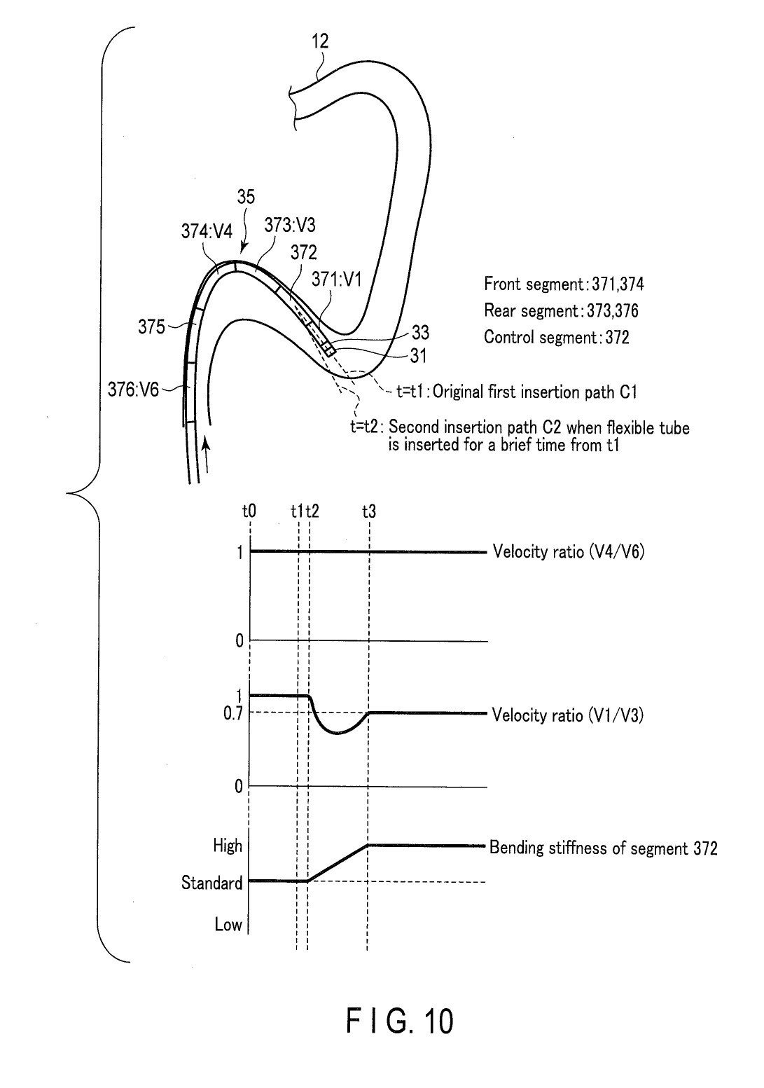

In the fifth example of the control shown in FIG. 10, it is assumed that since the bending stiffness of the segment 371 is insufficient at the time t2 with respect to the time t1, the segment 371 is bent toward the center direction of the bending R of the segment 371. In the fifth example, the target value of the velocity ratio is different from that in the first example. In the fifth example, it is assumed that the input device 90 sets the threshold value of the velocity ratio to 1, sets the target value of the velocity ratio to 0.7, and sets the control start mode to the automatic start mode. It is also assumed that the tube portion 12 has a single bent portion.

t=t1: The insertability determinator 85 determines that since the velocity ratio (V1/V3) is the same as the threshold value, the insertability is not decreased.

t=t2: The insertability determinator 85 determines that since the velocity ratio (V1/V3) is less than 1, which is the threshold value, the insertability is decreased, and then outputs the determination result to the stiffness controller 87. When the determination result is input, the stiffness controller 87 immediately increases the bending stiffness of the segment 372 so that the velocity ratio (V1/V3) becomes 0.7, which is the target value. Specifically, the stiffness controller 87 controls the variable stiffness portion 60 arranged in the segment 372 so as to increase the bending stiffness of the segment 372. By this control, the segment 372 becomes hard. Asa result, the segment 371 returns from the second insertion path C2 to the first insertion path C1, and thus the insertability of the flexible tube 35 is improved.

t=t3: Since the velocity ratio (V1/V3) has become 0.7, which is the target value, the stiffness controller 87 controls the variable stiffness portion 60 arranged in the segment 372 so that the high bending stiffness of the segment 372 is maintained.

The fifth example may be applied to the second example as a modification. That is, in the modification, it is assumed that the target value of the velocity ratio is 0.7, and the segment 371 is bent in a direction opposite to the center direction of the bending R of the segment 371. In the modification, the stiffness controller 87 operates in the same manner as in the second example.

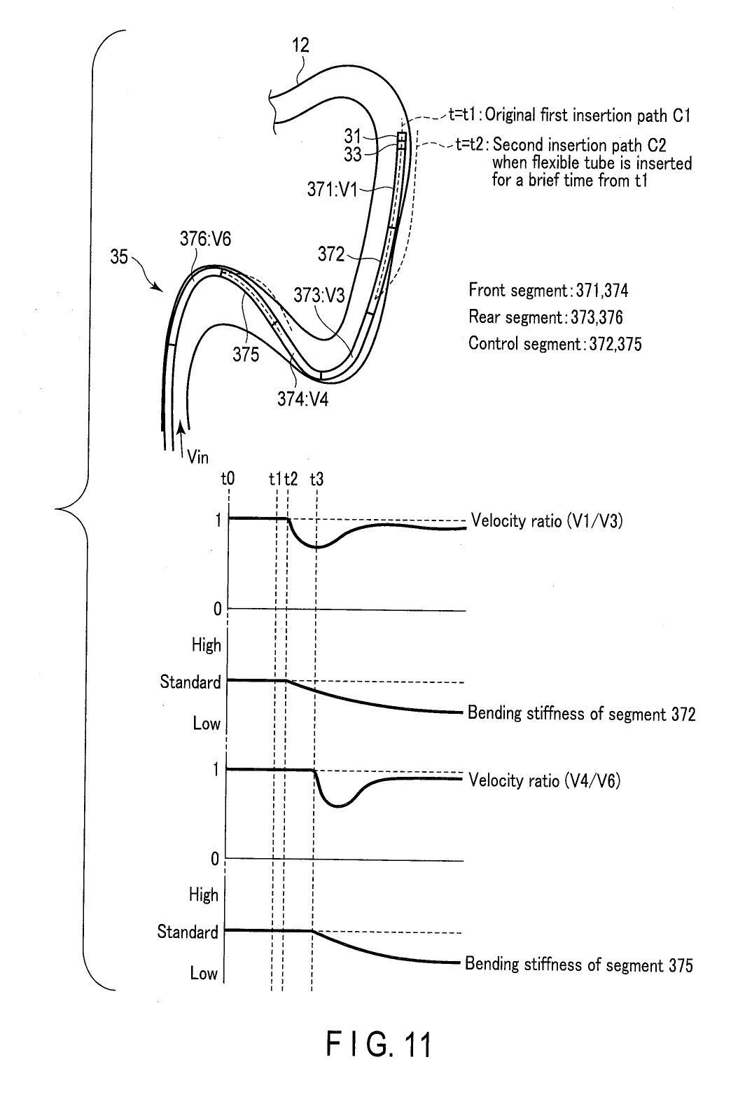

In the sixth example of the control shown in FIG. 11, it is assumed that since the bending stiffness of the segment 371 is excessive at the time t2 with respect to the time t1, the segment 371 is bent in a direction opposite to the center direction of the bending R of the segment 371. It is also assumed that since the bending stiffness of the segment 374 is excessive at a time t3 with respect to the time t2, the segment 374 is bent in a direction opposite to the center direction of the bending R of the segment 374. In the sixth example, it is assumed that the input device 90 sets the threshold value of the velocity ratio to 1, sets the target value of the velocity ratio to 1, and sets the control start mode to the automatic start mode. It is also assumed that the tube portion 12 has two bent portions.

t=t1: The insertability determinator 85 determines that since the velocity ratios (V1/V3, V4/V6) are the same as the threshold value, the insertability is not decreased.

t=t2: The insertability determinator 85 determines that since the velocity ratio (V1/V3) is less than 1, which is the threshold value, the insertability is decreased, and that the velocity ratio (V4/V6) is 1, which is the threshold value, and then outputs the determination result to the stiffness controller 87. When the determination result is input, the stiffness controller 87 immediately decreases the bending stiffness of the segment 372 so that the velocity ratio (V1/V3) becomes 1, which is the target value. Specifically, the stiffness controller 87 controls the variable stiffness portion 60 arranged in the segment 372 so as to decrease the bending stiffness of the segment 372. By this control, the segment 372 becomes soft. Asa result, the segment 371 returns from the second insertion path C2 to the first insertion path C1, and thus the insertability of the flexible tube 35 is improved.

t>t2: If the velocity ratio (V1/V3) would not become 1, which is the target value, due to the insertion environment, the velocity ratio is only required to be close to the target value. Accordingly, after the time t2, the stiffness controller 87 controls the variable stiffness portion 60 arranged in the segment 372 so as to cause the velocity ratio to be close to 1, which is the target value.

t=t3: The insertability determinator 85 determines that since the velocity ratio (V4/V6) is less than 1, which is the threshold value, the insertability is decreased, and then outputs the determination result to the stiffness controller 87. When the determination result is input, the stiffness controller 87 immediately decreases the bending stiffness of the segment 375 so that the velocity ratio (V4/V6) becomes 1, which is the target value. Specifically, the stiffness controller 87 controls the variable stiffness portion 60 arranged in the segment 375 so as to decrease the bending stiffness of the segment 375. By this control, the segment 375 becomes soft.

t>t3: If the velocity ratio (V4/V6) would not become 1, which is the target value, due to the insertion environment, etc., the velocity ratio is only required to be close to the target value. Accordingly, after the time t3, the stiffness controller 87 controls the variable stiffness portion 60 arranged in the segment 375 so as to cause the velocity ratio to be close to 1, which is the target value.

Next, working effects of the present embodiment will be described using the first example of the control shown in FIG. 7.

After the insertion apparatus 10 starts driving, the operator sets and inputs, through the input device 90, the segments 371 and 373 as the front segment and the rear segment, sets the threshold value of the velocity ratio to 1, sets the target value of the velocity ratio to 1, and sets the control start mod to the automatic start mode. From the time t0 to the time t1, the insertion section 30 including the flexible tube 35 is inserted into the tube portion 12, and is further inserted toward the deeper portion. The operator, through the input device 90, outputs the operation start instruction to the state detector 70, the state calculator 81, the velocity ratio calculator 83, and the insertability determinator 85. Then, the state detector 70 always detects the state information of the flexible tube 35, and the state calculator 81 always calculates the insertion paths C1 and C2 and the velocities V1 and V3 along the central axis of the flexible tube 35. In addition, the velocity ratio calculator 83 always calculates the velocity ratio (V1/V3) based on the velocities V1 and V3 calculated by the state calculator 81, and the insertability determinator 85 always determines whether the insertability is decreased. The velocities V1 and V3 are velocities along the central axis of the insertion path.

In an initial stage of the insertion indicating from the time t0 to the time t1, resistance of the tube portion 12 with respect to the flexible tube 35 is small, and the velocity ratio (V1/V3) is 1, which is the target value.

It is assumed that, at the time t2, by the operator's push operation, the flexible tube 35 has passed through the bent portion, the insertion environment has changed, and the resistance has increased. Then, it is assumed that the second insertion path C2 calculated by the state calculator 81 at the time t2 has been changed with respect to the original first insertion path C1 calculated by the state calculator 81 at the time t1, due to the resistance.

At the time t2, the state calculator 81 calculates that the segment 371 has been bent toward the center direction of the bending R of the segment 371 with respect to the first insertion path C1 due to a change in insertion environment, based on the change in the second insertion path C2 with respect to the first insertion path C1. When the segment 371 is bent toward the center direction of the bending R of the segment 371 with respect to the first insertion path C1 due to the change in insertion environment, the velocity ratio is lowered due to this bending. The reason for this is that, in a state where the control of the stiffness controller 87 has not yet been carried out at the times t1 and t2, the bending stiffness of the segment 371 at the time t2 is lower than the bending stiffness of the segment 371 arranged along the first insertion path C1 at the time t1 due to the change in insertion environment.

Accordingly, the insertability determinator 85 determines that the insertability of the flexible tube 35 from the current position to the deeper portion is decreased, based on the velocity ratio and the threshold value. Then, the insertability determinator 85 outputs the determination result to the stiffness controller 87.

The stiffness controller 87 increases the bending stiffness of the segment 372 so that the velocity ratio (V1/V3) becomes 1, which is the target value. By this control, the segment 372 becomes hard. Due to this change of the segment 372, the segment 371 arranged in front of the segment 372 is bent in a direction opposite to the center direction of the bending R of the segment 371. As a result, the velocity ratio gradually approaches the target value (for example, 1), and eventually reaches the target value (for example, 1). That is, the segment 371 arranged along the second insertion path C2 at the time t2 is arranged along the first insertion path C1 at the time t3. Namely, at the time t3, the segment 371 is inserted in a state of being arranged along the first insertion path C1 without deviating from the first insertion path C1. Since the velocity ratio (V1/V2) has become 1, which is the target value at the time t3, the stiffness controller 87 maintains the high bending stiffness of the segment 372.

When the bending stiffness of the segment 372 is controlled according to the velocity ratio as described above, the segment 371 is arranged along the first insertion path C1 and is inserted along the first insertion path C1 without deviating from the first insertion path C1. As a result, the flexible tube 35 is further inserted toward the deeper portion along the shape of the tube portion 12, and the insertability of the flexible tube 35 is improved.

In the present embodiment, the stiffness controller 87 controls the bending stiffness of the flexible tube 35 in units of segments based on a velocity ratio calculated based on velocity information. Accordingly, even if the flexible tube 35 is unintentionally bent due to the change in insertion environment and deviates from the original first insertion path C1, the flexible tube 35 is arranged along the original first insertion path C1 by the controlled bending stiffness, and is inserted along the original first insertion path C1. As a result, in the present embodiment, the insertability into the deeper portion of the tube portion 12 can be improved.

In the present embodiment, the stiffness controller 87 always controls the bending stiffness of the flexible tube 35 based on the velocity ratio. Therefore, even if the flexibility of the large intestine varies depending on the condition of the patient, or for each patient, and even if the insertion environment changes, good insertability can be provided.

When, in a state where the bending stiffness is not controlled, an insertion force is applied to the flexible tube 35 at the time t2, the insertion force would be converted by the bending stiffness at this time into, for example, a force that pushes up a large intestine wall of the large intestine. As a result, the large intestine wall is pushed up, the flexible tube 35 unintentionally gives an excessive load to the large intestine wall, and the patient feels pain. However, in the present embodiment, the flexible tube 35 is arranged along the original first insertion path C1 by the controlled bending stiffness, and is inserted along the original first insertion path C1. Thus, even if the insertion force is applied to the flexible tube 35, the insertion force is used as a propulsion force, and the flexible tube 35 propels without giving an excessive load to the large intestine wall. Therefore, the flexible tube 35 does not unintentionally give an excessive load to the large intestine wall, and the patient's pain is reduced. Furthermore, in the present embodiment, it is possible to prevent the flexible tube 35 from buckling, and to reduce a load to be applied to the insertion target without unintentionally giving an excessive load to a wall of the tube portion 12.

In the present embodiment, the bending stiffness is controlled in each segment 37. Thus, in the present embodiment, the bending stiffness of the flexible tube 35 can be precisely controlled.

In the present embodiment, the velocity ratio calculator 83 calculates a velocity ratio, and the insertability determinator 85 determines whether the insertability is decreased based on the velocity ratio and a threshold value. The stiffness controller 87 always controls the bending stiffness of the flexible tube 35 according to the determination result determined based on the velocity ratio. Accordingly, a responsiveness (followability) of the distal end of the flexible tube 35 with respect to the hand side of the flexible tube 35 can be improved, and the operability for insertion can be improved.

In the present embodiment, the input device 90 sets a front segment and a rear segment, which are separated from each other. In the present embodiment, therefore, the velocity ratio can be certainly calculated, the responsiveness of the flexible tube 35 with respect to the hand side of the flexible tube 35 can be certainly improved, and the operability for insertion can be certainly improved.

In the present embodiment, the stiffness controller 87 determines the bending stiffness of the flexible tube 35 through the variable stiffness portion 60 immediately after the insertability determinator 85 determines that the insertability of the flexible tube 35 is decreased. Thus, it is possible to immediately correct the decreased insertability, and always provide good insertability.

Alternatively, in the present embodiment, when the insertability determinator 85 determines that the insertability of the flexible tube 35 is decreased and the input device 90 inputs the control start instruction to the stiffness controller 87, the stiffness controller 87 controls the bending stiffness of the flexible tube 35 through the variable stiffness portion 60. Therefore, it is possible to correct the decreased insertability according to determination of the operator, and to provide good insertability at a timing desired by the operator.

In the present embodiment, the bending stiffness of the control segment 37i arranged between the front segment and the rear segment is controlled so that a velocity ratio becomes a target value of the velocity ratio. Thus, the bending stiffness of the flexible tube 35 can be precisely controlled. The control segment 37i may be all segments 37 arranged between the front segment and the rear segment, or may be a segment 37 selected by the input device 90 from among the segments 37 arranged between the front segment and the rear segment. Thus, a site where the bending stiffness of the flexible tube 35 is to be controlled can be set as desired, and good insertability can be provided according to the insertion environment.

The stiffness controller 87 according to the present embodiment may, before controlling the bending stiffness of the control segment 37i based on a determination result of the insertability determinator 85, search for a control condition for the control of the bending stiffness, and control the bending stiffness in units of segments based on the searched control condition. For this purpose, the stiffness controller 87 varies a value of the bending stiffness with a lapse of time, and associates the value of the bending stiffness with a velocity ratio in a variation process. The temporal variation of the value of the bending stiffness shows a constant cycle, such as a sine wave. As for this temporal variation, it may be the elapse of at least one cycle. The stiffness controller 87 searches for a tendency of the velocity ratio corresponding to the bending stiffness in the variation process. The stiffness controller 87 sets a control condition from the searched tendency, and controls the bending stiffness under the set control condition. This will be described as the seventh example of the control below.

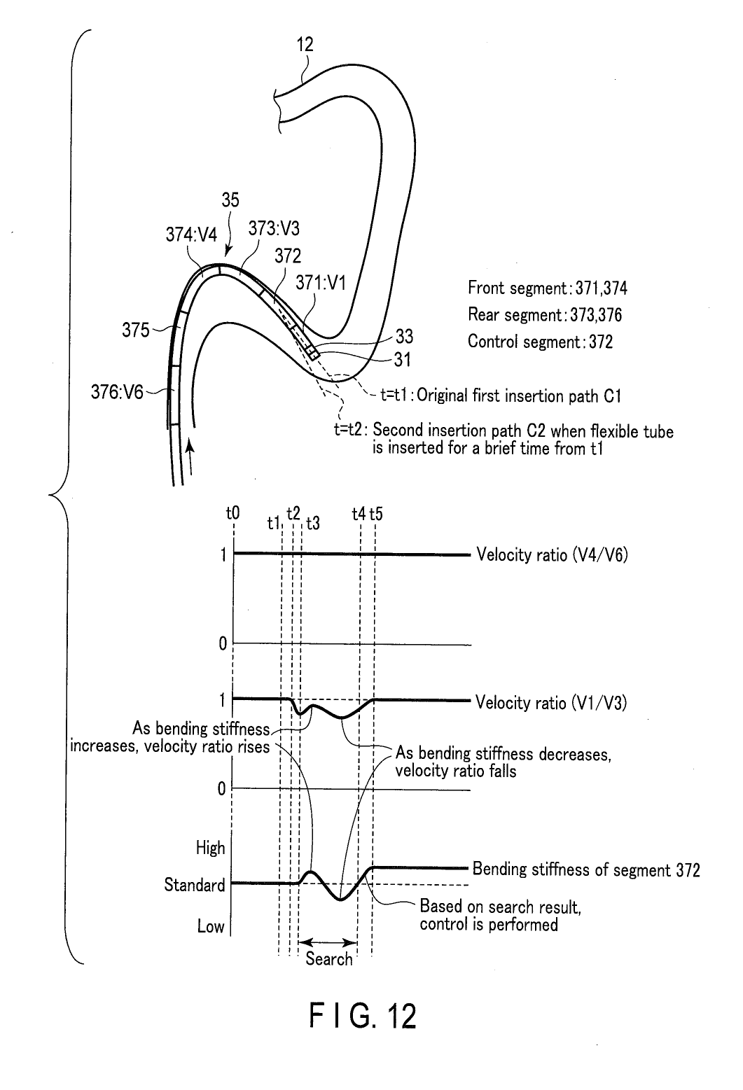

In the seventh example of the control shown in FIG. 12, it is assumed that since the bending stiffness of the segment 371 is insufficient at the time t2 with respect to the time t1, the segment 371 is bent toward the center direction of the bending R of the segment 371. In the seventh example, it is assumed that the input device 90 sets the threshold value of the velocity ratio to 1, sets the target value of the velocity ratio to 1, and sets the control start mode to the automatic start mode. It is also assumed that the tube portion 12 has a single bent portion.

t=t1: The insertability determinator 85 determines that since the velocity ratio (V1/V3) is the same as the threshold value, the insertability is not decreased.

t=t2: The insertability determinator 85 determines that since the velocity ratio (V1/V3) is less than 1, which is the threshold value, the insertability is decreased, and then outputs the determination result to the stiffness controller 87.

t=t3 to t4: The stiffness controller 87 searches for a control condition by varying the value of the bending stiffness of the segment 372 sinusoidally. Specifically, for searching, the stiffness controller 87 controls the variable stiffness portion 60 arranged in the segment 372 so that the value of the bending stiffness varies sinusoidally, for example. In this variation process, there is a tendency that the velocity ratio approaches the target value as the value of the bending stiffness is increased, and the velocity ratio departs from the target value as the value of the bending stiffness is decreased. Then, the stiffness controller 87 searches for a tendency, and sets a control condition that the velocity ratio approaches the target value from the tendency. Herein, the control condition is to increase the value of the bending stiffness.

t=t4: The stiffness controller 87 controls the bending stiffness under the set control condition. Specifically, the stiffness controller 87 controls the variable stiffness portion 60 disposed in the segment 372 so as to increase the bending stiffness. By this control, the segment 372 becomes hard.

t=t5: Since the velocity ratio (V1/V3) has become 1, which is the target value, the stiffness controller 87 controls the variable stiffness portion 60 arranged in the segment 372 so that the high bending stiffness is maintained.

As described above, in the present embodiment, the stiffness controller 87 controls the bending stiffness based on the searched control condition. Accordingly, the insertability into the deeper portion of the tube portion 12 can be certainly improved.

The present invention is not limited to the above embodiment as it is, and structural elements can be modified and embodied without departing from the gist thereof in the implementation stage. Furthermore, various inventions can be formed by appropriately combining structural elements disclosed in the above embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A flexible tube insertion apparatus comprising:
a flexible tube that is divided into segments arranged in series along a central axis and is to be inserted into an insertion target;
a variable stiffness portion that varies a bending stiffness of the flexible tube in units of segments;
a state detector that detects state information related to a state of the flexible tube including at least velocity information of the flexible tube;
a state calculator that calculates the velocity information of the flexible tube at two portions of the flexible tube based on the state information; and
a stiffness controller that controls the bending stiffness of the flexible tube in units of segments through the variable stiffness portion based on a velocity ratio calculated based on the velocity information of the two portions.

2. The flexible tube insertion apparatus according to claim 1, comprising a velocity ratio calculator that calculates the velocity ratio indicating a value obtained by dividing velocity information of a front segment arranged forward in an insertion direction of the flexible tube by velocity information of a rear segment arranged rearward in the insertion direction of the flexible tube.

3. The flexible tube insertion apparatus according to claim 2, comprising an insertability determinator that determines whether insertability of the flexible tube from a current position to a deeper portion is decreased based on the velocity ratio calculated by the velocity ratio calculator,
wherein the stiffness controller controls the bending stiffness in units of segments when the insertability determinator determines that the insertability of the flexible tube is decreased.

4. The flexible tube insertion apparatus according to claim 3, wherein the insertability determinator compares the velocity ratio with a threshold value to determine that the insertability of the flexible tube is decreased when the velocity ratio is less than the threshold value.

5. The flexible tube insertion apparatus according to claim 4, comprising an input device that sets the front segment and the rear segment, and further inputs the threshold value.

6. The flexible tube insertion apparatus according to claim 3, wherein the stiffness controller controls the bending stiffness of the flexible tube through the variable stiffness portion immediately after the insertability determinator determines that the insertability of the flexible tube is decreased.

7. The flexible tube insertion apparatus according to claim 3, comprising an input device that inputs a control start instruction of the stiffness controller to the stiffness controller,
wherein the stiffness controller controls the bending stiffness of the flexible tube through the variable stiffness portion, when the insertability determinator determines that the insertability of the flexible tube is decreased and the input device inputs the control start instruction to the stiffness controller.

8. The flexible tube insertion apparatus according to claim 3, wherein the stiffness controller controls a bending stiffness of each of one or more of the segments arranged between the front segment and the rear segment so that the velocity ratio becomes a target value of the velocity ratio.

9. The flexible tube insertion apparatus according to claim 8, comprising an input device that sets the target value to be a value equal to or less than 1.

10. The flexible tube insertion apparatus according to claim 3, wherein, based on the state information, the state calculator calculates whether the front segment is bent toward a center direction of bending of the front segment, or is bent in a direction opposite to the center direction of the bending of the front segment, and
wherein the stiffness controller
controls the bending stiffness of each of one or more of the segments arranged between the front segment and the rear segment to be high when the front segment is bent toward the center direction of the bending of the front segment, and
controls the bending stiffness of each of one or more of the segments arranged between the front segment and the rear segment to be low when the front segment is bent in the direction opposite to the center direction of the bending of the front segment.

11. The flexible tube insertion apparatus according to claim 3, wherein the stiffness controller searches for a control condition for control of the bending stiffness, and controls the bending stiffness in units of segments based on the searched control condition.

12. The flexible tube insertion apparatus according to claim 11, wherein the stiffness controller varies a value of the bending stiffness with a lapse of time, searches for a tendency of the velocity ratio corresponding to the bending stiffness in a variation process, sets the control condition from the searched tendency, and controls the bending stiffness under the set control condition.
